(19) United States
(12) Patent Application Publication
(10) Pub. No.: US 2024/0018581 A1
(43) Pub. Date: Jan. 18, 2024

(54) MIXTURE DECONVOLUTION METHOD FOR IDENTIFYING DNA PROFILES

(71) Applicant: Massachusetts Institute of Technology, Cambridge, MA (US)

(72) Inventors: Philip Fremont-Smith, Newmarket, NH (US); Chelsea Lynn Lennartz, Hollis, NH (US); Natalie Damaso, Woburn, MA (US)

(73) Assignee: Massachusetts Institute of Technology, Cambridge, MA (US)

(21) Appl. No.: 18/197,641

(22) Filed: May 15, 2023

Related U.S. Application Data
(60) Provisional application No. 63/389,748, filed on Jul. 15, 2022.

(51) Int. Cl. C12Q 1/6869; G16B 20/20; G16B 40/10; G16B 30/00

(52) U.S. Cl. CPC C12Q 1/6869; G16B 20/20; G16B 40/10; G16B 30/00

(57) ABSTRACT

This patent application relates generally to mixture deconvolution systems and methods for identifying DNA profiles. Various embodiments of the present invention concern the deconvolution of unknown DNA profiles in a two-person DNA mixture into two DNA profiles. Deconvolution methods isolate distinct DNA profiles from a DNA mixture without the need to match against DNA reference profiles. Various embodiments include a mixture deconvolution pipeline that involves a series of mathematical steps and machine learning algorithms to achieve the desired performance and decision-support outputs. Various embodiments enable distant familial matching to existing investigative genetic genealogy (IGG; also known as forensic genetic genealogy (FGG)) databases. This capability enables the generation of investigative leads from unresolved casework samples (i.e., DNA mixtures) by identifying possible genealogical relationships to one or more person(s) of interest. Such aspects may be performed in association with one or more systems used for genetic identification.

### RELATED APPLICATIONS

This application claims priority under 35 U.S.C. § 119(e) to U.S. Provisional Application Ser. No. 63/389,748, filed Jul. 15, 2022, and entitled “MIXTURE DECONVOLUTION METHOD FOR IDENTIFYING DNA PROFILES,” which is incorporated herein by reference in its entirety for all purposes.

### GOVERNMENT SUPPORT

This invention was made with government support under FA8702-15-D-0001 awarded by the U.S. Air Force. The government has certain rights in the invention.

### BACKGROUND

This patent application relates generally to mixture deconvolution systems and methods for identifying DNA profiles.

Investigative genetic genealogy (IGG) has emerged as a new, rapidly growing field of forensic science since its use in identifying the Golden State Killer in 2018. Recent IGG techniques have had a significant impact on the resolution of current and, especially, cold criminal cases. As a result, IGG is in high demand across the international forensic community. Currently, IGG searches are conducted only with a single-source DNA profile, requiring the deconvolution of any DNA mixtures prior to its use for long-range familial searching. However, estimates indicate that ˜50% of forensic casework samples are low level, partially degraded and/or mixtures, leaving samples from unidentified human remains, violent crime and matters of national security unresolved. For example, forensic casework samples may include DNA mixtures from more than one person. Mixtures of the DNA of people who did not match reference database profiles (a significant fraction of DNA evidence) cannot be used for emerging/advanced methods like IGG by existing systems.

### SUMMARY OF THE INVENTION

It is appreciated that there is a need for a system and method to isolate distinct DNA profiles from a DNA mixture to enable searching in existing genealogy databases. Various embodiments described herein concerns the deconvolution of unknown DNA profiles in a two-person DNA mixture into two DNA profiles. Deconvolution methods isolate distinct DNA profiles from a DNA mixture without the need to match against DNA reference profiles. As provided herein, a system and method is provided for a mixture deconvolution pipeline that involves a series of mathematical steps and machine learning algorithms to achieve the desired performance and decision-support outputs. Various embodiments enable distant familial matching to existing investigative genetic genealogy (IGG; also known as forensic genetic genealogy (FGG)) databases. This capability enables the generation of investigative leads from unresolved casework samples (i.e., DNA mixtures) by identifying possible genealogical relationships to one or more person(s) of interest. Such aspects may be performed in association with one or more systems used for genetic identification.

According to some embodiments, aspects relate to addressing a large unmet need in the forensic genomics market: the ability to deconvolve DNA profiles of unknown persons that are mixed with DNA from one or more other person(s) to enable searching in existing genealogy databases. Adding this capability will improve the generation of investigative leads in challenging defense, intelligence, and prosecutorial cases which often rely on incomplete DNA profile reference databases that hamper case resolution as well as offer an additional revenue stream for commercial laboratories involved in the forensic industry.

In some embodiments described herein, a two-person mixture may be processed in such a manner that does not require reference DNA from a subject. Rather, processing of the mixture as well as one or more existing genealogical databases are used to identify an individual. This process is beneficial, as reference DNA is not required for identification. Rather, long-range familial searching may be used for determining investigative leads. Further, in some embodiments, machine learning methods may be applied to more accurately predict the sex of particular contributors. Such elements may be used in an overall identification strategy and identification pipeline.

Some embodiments include a series of mathematical steps and mechanized processes to ingest, process, and produce results (e.g., in the current standard Verogen's ForenSeq Kintelligence sequencing format) of a two-person mixture. During processing, multiple algorithms are applied to select two-person mixtures for evaluation; to identify contributor's sex and concentration; and finally, to deconvolve each Single Nucleotide Polymorphisms (SNP) profile. Such algorithms (and companion software implementation) may be, according to various embodiments, specifically designed to yield the predicted number of contributors (NOC) in the mixture as well as the estimated percent contribution, predicted sex, and predicted DNA profile of each contributor. This information may be used to compare the individual DNA profile of each contributor to a wide variety of genealogical databases.

According to one aspect, a system is provided. The system comprises a component configured to analyze an input DNA mixture comprising at least two DNA contributors, a component configured to identify the number of contributors in the DNA mixture, a component configured to identify the sex of the two DNA contributors, a component to estimate the concentration of the two DNA contributors, and a component adapted to determine an individual DNA profile for the two DNA contributors.

According to one embodiment, one or more forensic genealogy databases comprise DNA markers enabling long-range familial searching of at least three degrees. According to one embodiment, the system further comprises a supervised learning model, the model being trained on a plurality of classification features relating to the input DNA mixture. According to one embodiment, the plurality of classification features comprises at least one of a group comprising a plurality of autosomal loci of an existing panel, estimated concentrations for minor and major contributors, minor allele counts ratio for each autosomal loci within the input DNA mixture, number of loci with a minor allele within the input DNA mixture, and global allele frequencies for each of the plurality of autosomal loci of an existing panel. For example, a commercially available panel (e.g., commercially available from Verogen, Inc. or other sources) may be used that provides autosomal loci information.

According to one embodiment, the system further comprises applying a threshold responsive to a predicted DNA marker at each genetic location and the estimated concentrations. According to one embodiment, the supervised learning model includes a random forest model. According to one embodiment, the random forest model is operated to deconvolve two-person mixtures. According to one embodiment, the processing component is used within an identification pipeline. According to one embodiment, the processing component is used to identify and select two-person mixtures for processing through the identification pipeline. According to one embodiment, the supervised learning model includes at least one output from a group comprising a probability for each possible genotype combination contained in the mixture, a predicted genotype with the highest probability score, and predicted DNA profiles and corresponding prediction probabilities for each of the two DNA contributors. According to one embodiment, the processing component is configured to deconvolve input DNA mixture comprising two DNA contributors into two distinct DNA profiles. According to one embodiment, the processing component is configured to determine the two distinct DNA profiles without performing a comparison with one or more DNA reference profiles. According to one embodiment, the component configured to identify the sex of the two DNA contributors further comprises a learning model, the model being trained on a plurality of classification features relating to the input DNA mixture. According to one embodiment, the plurality of classification features comprises a total number of counts of non-autosomal loci of the input DNA mixture at each sex genetic location.

Still other aspects, examples, and advantages of these exemplary aspects and examples, are discussed in detail below. Moreover, it is to be understood that both the foregoing information and the following detailed description are merely illustrative examples of various aspects and examples and are intended to provide an overview or framework for understanding the nature and character of the claimed aspects and examples. Any example disclosed herein may be combined with any other example in any manner consistent with at least one of the objects, aims, and needs disclosed herein, and references to “an example,” “some examples,” “an alternate example,” “various examples,” “one example,” “at least one example,” “this and other examples” or the like are not necessarily mutually exclusive and are intended to indicate that a particular feature, structure, or characteristic described in connection with the example may be included in at least one example. The appearances of such terms herein are not necessarily all referring to the same example.

### BRIEF DESCRIPTION OF DRAWINGS

Various aspects of at least one example are discussed below with reference to the accompanying figures, which are not intended to be drawn to scale. The figures are included to provide an illustration and a further understanding of the various aspects and examples are incorporated in and constitute a part of this specification but are not intended as a definition of the limits of a particular example. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. In the figures:

FIG. 1 shows a process for identifying individuals in a mixture according to various embodiments;

FIG. 2 shows a matching component according to various embodiments;

FIG. 3 shows an example learning model according to various embodiments;

FIG. 4A-4B shows an example pipeline used to identify individuals from a two-person mixture according to various embodiments;

FIG. 5 shows example mixture deconvolution results showing sex prediction accuracy and percent of DNA deconvolved according to various embodiments;

FIG. 6 shows a simulated number of loci with a minor allele per Number of Contributors (NOC) according to various embodiments; and

FIG. 7 shows an example of thresholds selected for each genotype based on a prediction of tradeoffs according to various embodiments.

### DETAILED DESCRIPTION

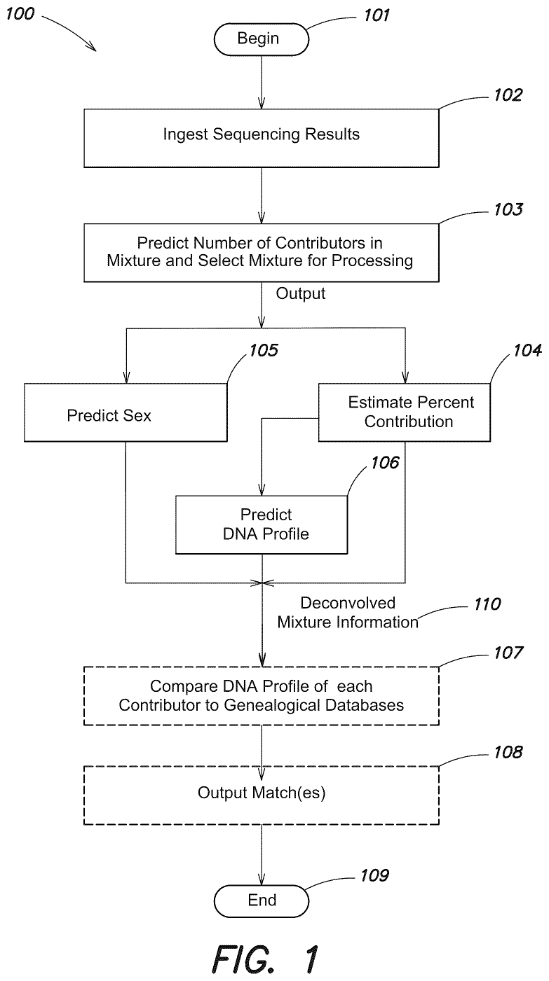

FIG. 1 shows a process 100 for identifying individuals in a mixture according to various embodiments. At block 101, process 100 begins. At block 102, the system ingests sequencing results, such as provided by a DNA sequencing system. For instance, a forensic mixture may be sequenced, and the information may be provided to a processor for identification. Process 100 may be performed as part of a larger identification pipeline.

At block 103, the system predicts a number of contributors within and selects a two-person mixture for processing. Further, the system may perform a number of processes by one or more components that process the mixture to determine predictions about the mixture. For example, the system may include a component (e.g., component 105) that is configured to predict a sex of one or more of the contributors. Further, the system may include a component (e.g., component 104) that is configured to estimate a percent contribution of the contributors to the mixture. Also, the system may include a component (e.g., component 106) that is configured to predict a DNA profile of the contributors. This deconvolved mixture information 110 may be then provided as outputs. The output information may be provided, for example, to a system that allows for identification of individuals identified from information determined from the deconvolved mixtures.

For example, as an optional set of steps, the information determined from deconvolving the mixture (e.g., deconvolved mixture information 110) may be used by an identification system to determine one or more output matches. For example, at block 107, the system compares a DNA profile of each contributor to one or more genealogical databases. At block 108, the system outputs any matches, and at block 109, process 100 ends.

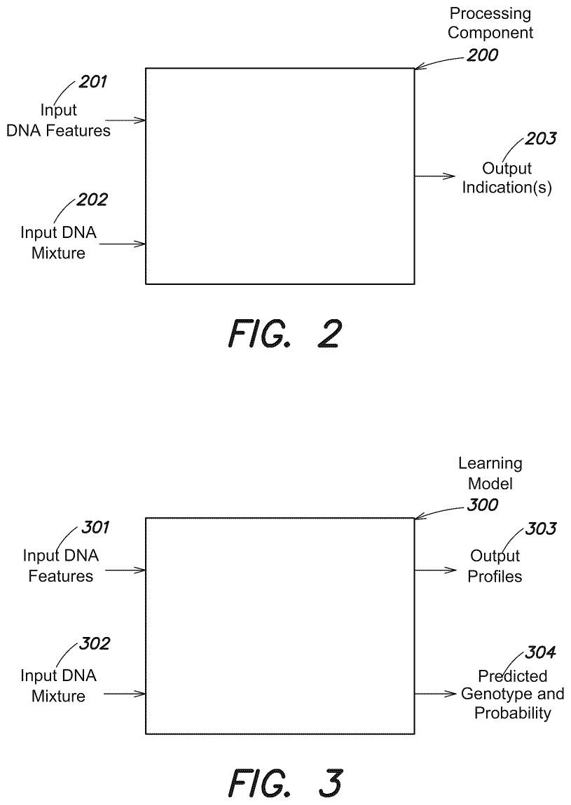

As discussed above and in further detail below, the system may be capable of processing an input DNA mixture and deconvolving information relating the mixture using input DNA features. In particular, FIG. 2 shows a processing component 200 according to various embodiments, processes one or more input DNA features 201 and an input DNA mixture 202 and produces one or more output indications 203. For instance, the system may provide output indication(s) that are deconvolved information relating to the individual DNA information relating to the contributors present within the input mixture.

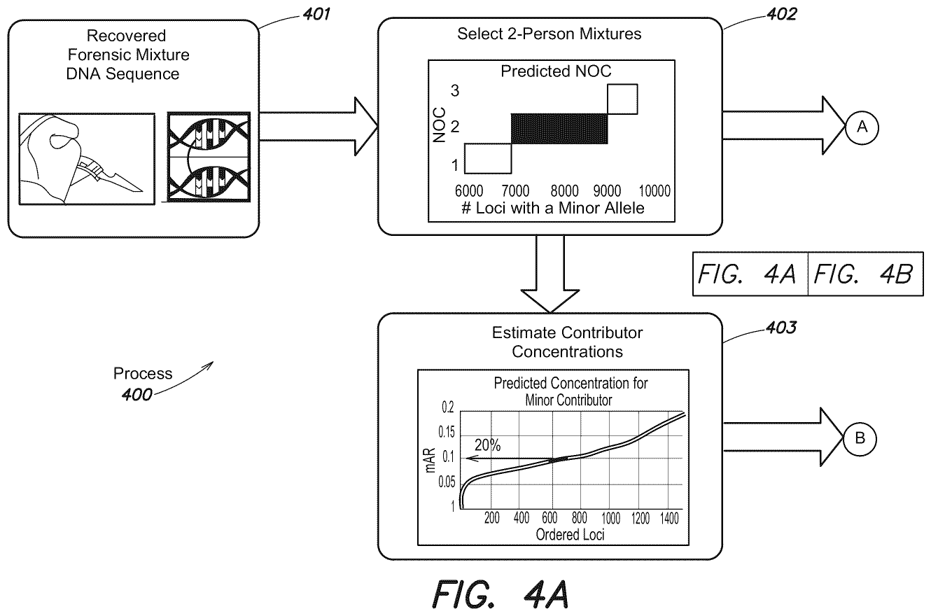

Further, as discussed above, system 200 may implement machine learning models (e.g., learning model 300) that provides information relating to individuals having DNA present in the input mixture. FIG. 3 shows an example learning model 300 according to various embodiments. In one example, learning model 300 may process one or more input DNA features 301 and one or more input DNA mixture 302 and produce as a result, one or more output profiles 303 of one or more contributors, and for each of these contributors, determine a predicted genotype and probability 304.

### Detailed Implementation

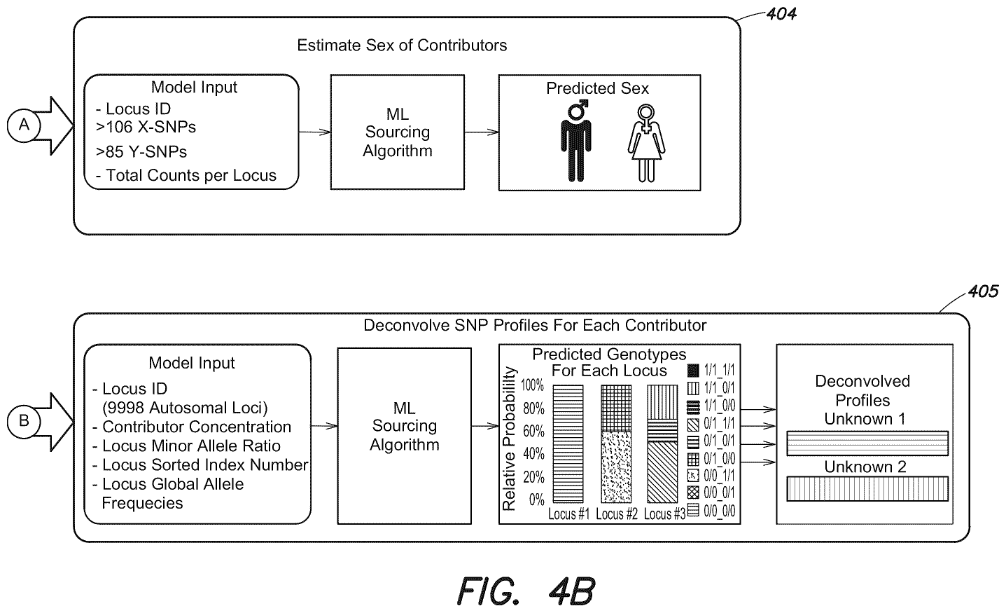

Some embodiments include a series of mathematical steps and mechanized processes to ingest, process, and produce results (e.g., in the current standard Verogen ForenSeq Kintelligence sequencing format) of a two-person mixture. During processing, multiple algorithms are applied to select two-person mixtures for evaluation; to identify contributor's sex and concentration; and finally, to deconvolve each Single Nucleotide Polymorphisms (SNP) profile. Such algorithms (and companion software implementation) may be, according to various embodiments, specifically designed to yield the predicted number of contributors (NOC) in the mixture as well as the estimated percent contribution, predicted sex, and predicted DNA profile of each contributor (e.g., as shown in FIG. 4A-4B). This information may be used to compare the individual DNA profile of each contributor to a wide variety of genealogical databases.

Various algorithms discover and refine unknown profiles from forensic DNA mixtures such as the apex unknown method, the unknown coalesce method, the SCOPE method, and direct deconvolution using a random forest classifier. Additional features of some embodiments of the present invention beyond these algorithms extract unknown DNA profiles from a two-person mixture include:
1. A sex determination step in the algorithm sequence, which provides additional contributor information that is valuable for investigative leads.
2. A novel machine learning algorithm was developed that predicts the DNA profile of each contributor with sufficiently high performance (high number of accurate DNA markers (n>3000)) to enable long-range familial searching (e.g., 3-4^{th} degree) in genetic genealogy databases. In some embodiments, the threshold tests described in the apex unknown method and the unknown coalesce method may be changed to a random forest supervised machine learning model (or other model type). New classification features may be used such as contributor concentrations, total number of minor allele calls in the mixture, and global allele frequencies for each autosomal genetic location from the Genome Aggregation Database (gnomAD), which provides additional information to increase performance.
3. Instead of the machine learning default predictions, custom thresholds may first be applied based on predicted DNA marker at each genetic location and contributor concentrations for that mixture sample, to thereby enable an assignment based on probabilities of potential pairings instead of a hard, binary 0-1 assignment to increase sensitivity and specificity.

In addition, a small portion of the SCOPE method such as the exemplary Equation described below may be leveraged to determine the number of contributors in the DNA mixture and the Unknown Concentration Estimation (UCE) method may be leveraged to determine the contributor concentrations of each individual in the mixture.

Exemplary Equation:

Number of loci with a minor allele=L−Σ(p²)^{N}
L=Number of Loci in Panel
N=Number of Contributors
p=Average Major Allele Frequency.

However, adaptations may be required for its compatibility with the Forenseq Kintelligence sequencing panel that had ˜10,000 DNA markers. In-silico mixtures may be modelled to calculate the expected mean number of minor alleles for a two-person mixture and the minor contributor's average mAR plateau to compare against the unknown mixtures to estimate the number of contributors and contributor concentrations, respectively. In some embodiments, the adapted number of contributor's algorithm and contributor concentration's algorithm may be sequentially processed and may be crucial first steps in one embodiment that 1) identifies and selects two-person mixtures for continuation through the deconvolution pipeline and 2) estimates the contributors' concentrations that is utilized as an input feature in the mixture deconvolution algorithm.

For mixture deconvolution, a random forest model may be used to deconvolve two-person mixtures using actual or estimated contributions (provided by the contributor concentrations algorithm mentioned above), minor allele ratio (mAR) at each autosomal genetic location, rank order of genetic locations as determine by the mAR, total number of minor allele calls in the mixture, and global allele frequencies for each autosomal genetic location from the Genome Aggregation Database (gnomAD). This algorithm may be specific to two-person mixtures using the Verogen ForenSeq Kintelligence genetic panel. In some embodiments, the model may provide the predicted DNA markers for each genetic location with their corresponding probability score. Custom probability thresholds based on DNA markers and contributor concentrations may be used in some embodiments to remove predicted DNA markers below the threshold to increase performance (specificity and sensitivity) relative to benchmark standards. For sex identification, a second random forest model is used to predict the sex of each contributor in an unknown two-person mixture. In some embodiments, the key classification feature employed by the model may be the total sequencing read count at each sex genetic location. This feature may be conveniently provided in the raw sequencing results from the instrument which is recorded in the standard Verogen ForenSeq Kintelligence sequencing format. This algorithm may be specific to two-person mixtures using the ForensSeq Kintelligence sex SNPs.

Recently, Investigative Genetic Genealogy (IGG) has been a rapidly growing forensic industry assisting in over 200 cold cases in the United States. IGG is currently conducted using single-source DNA profiles. Various embodiments of the present invention may have a high national security impact by providing the opportunity to utilize mixtures in addition to single-source profiles, thereby increasing the generation of investigative leads in challenging defense, intelligence, and prosecutorial cases. Beyond the national security impact, various embodiments of the present invention will fill a large gap in the forensic genomics market: the deconvolution of DNA profiles from a DNA mixture to enable searching of existing genealogy databases. In some embodiments, various aspects described herein may be incorporated within one or more computer systems for identifying individuals from one or more databases. In some embodiments, some aspects may be configured to operate within various software systems used to search various databases (e.g., Verogen's ForenSeq Kintelligence SNPs and GEDMatch database) implementing one or more workflows (e.g., Verogen's IGG workflow).

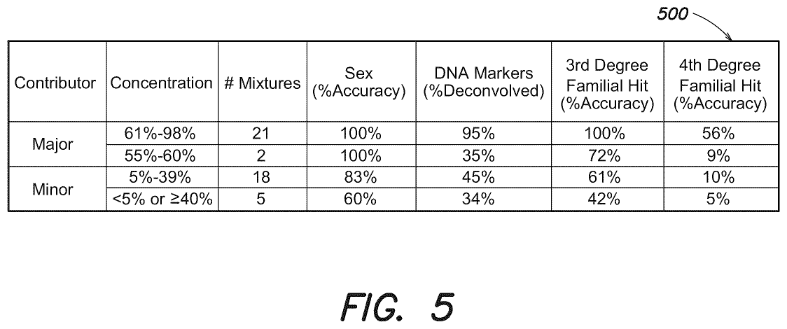

Various embodiments described herein have been demonstrated in a laboratory environment beyond proof-of-concept capability for two-person mixtures. Over 500 in silico and 30 real experimental mixtures (consisting of unblinded and blinded datasets) demonstrated feasibility and high performance, as shown in FIG. 5. For example, various embodiments of the present invention achieved 100% accuracy on identifying sex, deconvoluted 95% of the DNA markers, and achieved 100% and 56% accuracy in third degree and fourth degree familial hits respectively. The algorithms of some embodiments of the present invention may be packaged into a Docker container that can be easily transitioned to be utilized by any individual and machine.

Recent IGG techniques have had a significant impact on the resolution of current and, especially, cold criminal cases. As a result, IGG is in high demand across the international forensic community. Mixtures of the DNA of people who did not match reference database profiles (a significant fraction of DNA evidence) cannot be used for emerging/advanced methods like IGG by existing systems. Advantages of using various methods as described herein include the ability to identify the sex and recover DNA profiles for each unknown contributor of two-person mixtures to enable long-range familial searching (e.g., 3-4^{th} degree) in genetic genealogy databases. In addition, some embodiments described herein provide a probability value associated with the predicted DNA profiles that yield confidence scores for the deconvolved profiles. These capabilities provide a significant impact on the large fraction of cases where DNA mixtures currently prevent the use of IGG searches.

### Deriving a Predicted NOC for the Selected Two-person Mixture

As illustrated by FIG. 4A, some embodiments may begin using a recovered forensic mixture DNA sequence at block 401. At block 402, the number of contributors is estimated by counting the number of loci with a minor allele, determined as a minor allele ratio (mAR)>=0.01, in the mixture and comparing that number with a mean expected number of minor alleles for a two-person mixture. An expected number of minor alleles for up to 6 contributors may be calculated in some embodiments by:
randomly generating a predetermined number (e.g., 3500) of insilico mixtures from a predetermined number (e.g., 83) of DNA references representing up to six contributors;
calculating mAR for each autosomal loci using reference and alternate allele counts from the ForenSeq Kintelligence sequencing results text file;
leveraging the equation specified above to calculate the number of loci with a minor allele for each mixture;
computing, for each NOC (i.e., 1-6), mean and standard deviation regarding number of loci with a minor allele.

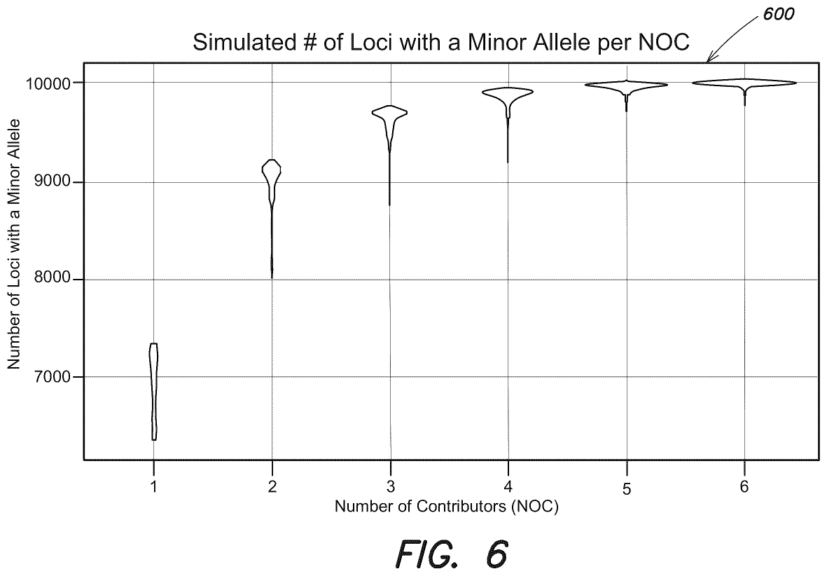

Summary statistics for each NOC based on simulation are listed in the table 1 below and the distribution can be visualized in FIG. 6.

TABLE 1

| NOC | Mean | SD |
|---|---|---|
| 1 | 6959 | 326 |
| 2 | 8988 | 279 |
| 3 | 9623 | 124 |
| 4 | 9843 | 76 |
| 5 | 9929 | 41 |
| 6 | 9968 | 21 |

In some embodiments, the NOC may be predicted for an unknown mixture by:
calculating the number of loci with a minor allele (mAR>=0.01);
computing, for all NOCs (i.e., 1-6), z-score using simulated mean and standard deviation for the corresponding NOC (Table 1);
selecting a NOC based on the lowest absolute z-score; and
selecting two person mixtures for continuation.

Table 2 below illustrates the computed z-scores for all possible NOCs from an unknown mixture having 8797 loci with a minor allele. The NOC of two resulted in the lowest absolute z-score predicting two contributors in the mixture.

TABLE 2

| NOC | 1 | 2 | 3 | 4 | 5 | 6 |
|---|---|---|---|---|---|---|
| Z-Score | 5.64 | 0.68 | 6.65 | 13.77 | 27.81 | 55.5 |

### Determining Sexes of Contributors

In some embodiments, a Random Forest model may be generated using a predetermined number (e.g., 500) of insilico mixtures and the total number of counts per non-autosomal locus (normalized to counts per million) to predict the sex of each contributor in an unknown two-person mixture. An exemplary process using exemplary model inputs is illustrated by block 404 of FIG. 4B.

In other embodiments, similar approaches described below for the deconvolution method may be implemented to determine the sex of the contributors (e.g., deterministic approaches using counts-based features, other probabilistic supervised machine learning methods). In some embodiments, a tier approach that first utilizes the y-sex markers to determine the presence/absence of male in the mixture and then determines the ratio of male to female presence utilizing the signal ratio of the y to x-sex markers.

In some embodiments, the model input may be the total number of counts per non-autosomal (sex) loci normalized to counts per million. Table 3 illustrates an exemplary model input illustrating normalized total counts for 3 non-autosomal loci including 233 non-autosomal loci in total.

TABLE 3

| Sex Loci | rs7520386 | rs1445225 | rs12750589 |
|---|---|---|---|
| Normalized Total Counts | 75 | 26 | 83 |
| (Counts/Million) | | | |

In some embodiments, the model output may be a single character vector representing the sex of the major/minor contributor (e.g., “F/M”). For example, “F/M” represents a mixture with a female major contributor and male minor contributor.

In some embodiments, sex markers (X and Y-SNPs) may be an effective method for determining the sex of an individual and estimating the ratio of male and female in a mixture. More specifically, the presence or absence of the Y chromosome is critical as only males will inherit a Y chromosome and will only have a single copy of the X chromosome.

### Thresholding

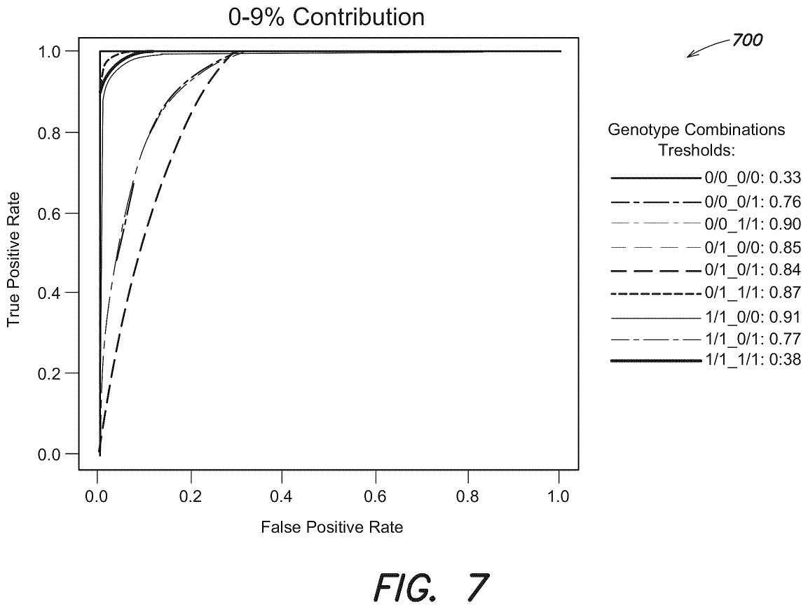

In some embodiments, relative probability thresholds may be selected for each genotype and contributor concentrations using 500 insilico mixtures representing various ethnicities and mixture contribution ratios. Optimized thresholds were determined by algorithmically decreasing the number of false positives genotype calls below a target (10% per possible genotype combination). This target was chosen to provide a sufficiently high number of true positive genotype calls (i.e., >3000 loci for 3rd degree relationship and >6000 for 4th degree relationship) for searching in IGG databases. FIG. 7 illustrates an example of the performance tradeoffs for each genotype combination from contributors with less than 9% contribution.

In some embodiments, the model output may provide the predicted genotypes for each loci with their corresponding probability score. Table 4 shows an example of threshold implementation in which the second row corresponds to genotype calls below the threshold that are assigned “./._./.” and the first row corresponds to assigned genotype calls.

In some embodiments genotype calls below the threshold (optimized threshold for given genotype combination and contributor concentration) are assigned “./.” rather than the predicted genotype to reduce the number of false positive rates. Genotype calls above the threshold may also be assigned. Table 4 provides an example of two genotype calls demonstrating both scenarios in which the predicted genotype of the first row is assigned based on probability score being above the threshold and in the second row, a predicted genotype is not called. FIG. 7 shows an example of thresholds selected for each genotype combination based on prediction trade-offs for contributors with <9%.

TABLE 4

| | Predicted | Probability | |
|---|---|---|---|
| LocusID | Genotype | Score | Result |
| rs10245106 | 1/1_1/1 | 0.96 | 1/1_1/1 |
| rs6989074 | 1/1_1/1 | 0.33 | ./._./. |

### Deconvolution of SNP profiles

Block 405 of FIG. 4B shows an exemplary process for deconvolving SNP profiles for each contributor according to various embodiments. In some embodiments, a Random Forest model may be generated using 500 insilico mixtures to provide deconvolved SNP profiles for each contributor in an unknown two-person mixture.

In other embodiments, other probabilistic classification methods could be utilized as well as a deconvolution method to extract unknown DNA profiles from a two-person mixture.

In some embodiments, The model input may include:
Locus_ID: a list of autosomal loci from the Verogen ForenSeq Kintelligence panel in which certain loci may be more important in distinguishing profiles.
Low_contrib/high_contrib: estimated concentrations for minor and major contributors. The contribution of each person is highly important in separating the profiles as the number of counts contributed at each loci is a direct relation to this value.
mAR: minor allele ratio (mAR) for each loci calculated by using reference and alternate allele counts from the ForenSeq Kintelligence sequencing results text file. The mAR is related to each person's genetic profile and DNA contribution amount to the mixture.
Order: rank order of loci as determined by mAR.
Num_mm: number of loci with a minor allele in the unknown mixture (mAR>=0.01) relating to the number of contributors (NOC) in a mixture.
gAF: global allele frequencies for each loci obtained from the Genome Aggregation Database (gnomAD). Certain SNPs have less frequently seen minor alleles which lends these loci more discriminatory power.

Table 5 shows an example of the 5 features used for model inputs, as explained above.

TABLE 5

| Locus_ID | mAR | order | low_contrib | high_contrib | num_mm | gAF |
|---|---|---|---|---|---|---|
| rs6690515 | 0.348706412 | 2839 | 0.06032029 | 0.9396797 | 8666 | 0.343133 |
| Rs28635343 | 0.005440469 | 1301 | 0.06032029 | 0.9396797 | 8666 | 0.188158 |
| Rs16824588 | 0.499372647 | 5189 | 0.06032029 | 0.9396797 | 8666 | 0.424881 |

In some embodiments, the model output may include (i) a probability for each possible genotype combination in the mixture; (ii) a predicted genotype (genotype with the highest probability score). Table 6 shows an exemplary model output illustrating the probability score for all possible genotype combination for each loci and the predicted genotype.

TABLE 6

| Locus_ID | 0/0_0/0 | 0/0_0/1 | 0/0_1/1 | 0/1_0/0 | 0/1_0/1 | 0/1_1/1 | 1/1_0/0 | 1/1_0/1 | 1/1_1/1 | pred |
|---|---|---|---|---|---|---|---|---|---|---|
| rs6690515 | 0.01 | 0.62 | 0.00 | 0.00 | 0.26 | 0.00 | 0.06 | 0.05 | 0.00 | 0/0_0/1 |
| rs28635343 | 0.68 | 0.00 | 0.00 | 0.31 | 0.00 | 0.00 | 0.01 | 0.00 | 0.00 | 0/0_0/0 |
| rs16824588 | 0.01 | 0.39 | 0.00 | 0.00 | 0.53 | 0.00 | 0.00 | 0.07 | 0.00 | 0/1_0/1 |

The utility of these features has been previously demonstrated to be valuable for deconvolving an unknown mixture.

### Major Components of the Software and the Functions

In some embodiments, the software may include code (e.g., written in R) which ingests and deconvolves the standard Verogen ForenSeq Kintelligence sequencing results text file. In some embodiments, files may be output for each of the two contributors containing the predicted number of contributors in the mixture as well as the estimated percent contribution, predicted sex, and predicted DNA profile of each contributor. In some embodiments, the files may then be used individually to compare the DNA profile of each contributor to genealogical databases. In some embodiments, the algorithm code may be packaged into a Docker container that can be easily transitioned to be utilized by any individual and machine.

In some embodiments, the object code may include sysdata as an RDA file, which may include one or more input files:
‘master_snps’: a list of DNA markers subsetted from the ForenSeq Kintelligence panel based upon consistent performance with corresponding global allele frequencies (gAF) from the Genome Aggregation Database (gnomAD);
‘rf_sex’: random forest model used in ‘detSex’ source code to predict the sex of the contributors; and
‘rf_deconvolve’: random forest model used in ‘deconvolve2p’ source code to deconvolve two person mixtures

In some embodiments, the source code may include:
‘read.verogen’: source code to read in the Verogen ForenSeq Kintelligence sequencing results text file, having inputs: 1) file name and 2) pathway of the mixture file to analyze, and output: dataframe with all data needed for mixture analysis
‘estnoc’: source code to estimate the number of contributors in the mixture, having input: mixture dataframe from ‘read.verogen’ and output: single integer with the number of contributors. Only two-person mixtures are accepted to continue to the other source codes.
‘estcontrib’: source code to estimate the contributions of each individual in a 2 person mixture, having input: mixture dataframe from ‘read.verogen’ and output: vector with the contribution estimates for the minor and major contributors
‘detSex’: source code to determine the sex of each individual in a 2 person mixture using random forest model having inputs: 1) mixture dataframe from ‘read.verogen’, 2) ‘rf_sex’ model from ‘sysdata’ and output: single character vector predicting the sex of each contributor (i.e., “M” “F” for high & low contributors, respectively)
‘deconvolve2p’: a source code to deconvolve a 2 person mixture using random forest model and custom thresholds based on DNA markers and contributor concentrations, having inputs: 1) mixture dataframe from ‘read.verogen’, 2) vector of contribution estimates from ‘estcontrib’, 3) ‘rf_deconvolve’ model from ‘sysdata’ and output: table with predicted DNA profiles and their corresponding prediction probabilities for each contributor
‘write.verogen’: source code to output a file for each of the two contributors containing the predicted number of contributors in the mixture, estimated percent contribution, predicted sex, and predicted DNA profile of each contributor, having input: 1) path where output file should be saved, 2) return values from: ‘estnoc’, ‘detSex’, ‘deconvolve2p’
‘run.verogen’: source code to run the deconvolution pipeline using the below above codes and files, having input: 1) source codes (i.e., read.verogen, estnoc, estcontrib, detSex, deconvolve2p, write.verogen), 2) sysdata.rda, 3) input mixture data and file path, and output: 1) txt file for each contributor with predicted genotype and probability value for each loci and 2) json file with NOC, percent contribution and sex for each contributor.

In some embodiments, one or more README files may include the steps needed to run the deconvolution pipeline (descriptions above) as well as how to build the Docker container. Table 7 shows an example of txt file output information that is generated for each contributor.

TABLE 7

| Locus_ID | Genotype | Predicted_Probability |
|---|---|---|
| rs731031 | 0/0 | 1 |
| rs868688 | 0/0 | 1 |
| rs12022636 | 0/0 | 1 |

Table 8 shows an example of output information.

TABLE 8

| Contributor | Minor | Major |
|---|---|---|
| Est_Num_Contributors | 2 | 2 |
| Est_Contribution_Percent | 6% | 94% |
| Sex | M | F |

Some embodiments include one or more of the following 3^{rd}-party dependencies:

| Name | Code Repository URL | License Type |
|---|---|---|
| R | https://cran.r-project.org/mirrors.html | GPL-2 \| |
| | | GPL-3 |
| tidyverse | https://www.tidyverse.org/packages/ | MIT |
| randomForest | https://cran.r-project.org/web/packages/randomForest/randomForest.pdf | GPL-2 \| |
| | | GPL-3 |
| Docker | https://github.com/docker | Apache- |
| | | 2.0 |
| | | License |
| Ubuntu | https://ubuntu.com/download | GPLv2 |
| Linux | | |
| apt-utils | https://github.com/Debian/apt | GPLv2+ |
| gpg | https://github.com/gpg/gnupg | GPLv3 |
| wget | https://www.gnu.org/software/wget/ | GPLv3 |
| curl | https://github.com/curl | Custom |
| | | (MIT- |
| | | like) |
| libcurl4 | https://github.com/curl | Custom |
| | | (MIT- |
| | | like) |
| libcurl4- | https://github.com/curl | Custom |
| openssl-dev | | (MIT- |
| | | like) |
| libxml2-dev | https://github.com/GNOME/libxml2 | MIT |
| openssl | https://github.com/openssl/openssl | Apache |
| | | License |
| | | v2 |
| libssl-dev | https://github.com/openssl/openssl | Apache |
| | | License |
| | | v2 |
| libsodium-dev | https://github.com/jedisct1/libsodium | ISC |
| zlib1g-dev | https://zlib.net/ | zlib |

| Name | License URL |
|---|---|
| R | https://www.r-project.org/Licenses/ |
| tidyverse | https://tidyverse.tidyverse.org/LICENSE.html |
| randomForest | https://cran.r-project.org/web/packages/randomForest/index.html |
| Docker | https://www.docker.com/legal/docker-software-end-user-license-agreement |
| Ubuntu | http://manpages.ubuntu.com/manpages/bionic/man7/gpl.7gcc.html |
| Linux | |
| apt-utils | http://changelogs.ubuntu.com/changelogs/pool/main/a/apt/apt_2.0.6/copyright |
| gpg | https://gnupg.org/ |
| wget | https://www.gnu.org/software/wget/ |
| curl | https://curl.se/docs/copyright.html |
| | https://curl.se/docs/faq.html#I_have_a_GPL_program_can_I_use |
| libcurl4 | https://curl.se/docs/copyright.html |
| | https://curl.se/docs/faq.html#I_have_a_GPL_program_can_I_use |
| libcurl4- | https://curl.se/docs/copyright.html |
| openssl-dev | https://curl.se/docs/faq.html#I_have_a_GPL_program_can_I_use |
| libxml2-dev | https://github.com/GNOME/libxm12/blob/master/Copyright |
| openssl | https://www.openssl.org/source/license.html |
| libssl-dev | https://www.openssl.org/source/license.html |
| libsodium-dev | https://launchpad.net/ubuntu/hirsute/+source/libsodium/+copyright |
| zlib1g-dev | https://en.wikipedia.org/wiki/Zlib_License |

In some embodiments, one or more of the third party dependencies are unmodified.

### Example Computer System

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be understood that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be understood that one implementation of the embodiments of the present invention comprises at least one non-transitory computer-readable storage medium (e.g., a computer memory, a portable memory, a compact disk, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be understood that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

What is claimed is:

1. A system comprising:
a processing component configured to process an input DNA mixture,
a component configured to identify the number of contributors in the DNA mixture and select mixtures comprising two DNA contributors;
a component configured to identify a sex of the two DNA contributors;
a component configured to identify a concentration of the two DNA contributors;
a component adapted to determine an individual DNA profile for the two DNA contributors.

2. The system according to claim 1, wherein the one or more forensic genealogy databases comprise DNA markers enabling long-range familial searching of at least three degrees.

3. The system according to claim 1, further comprising a supervised learning model, the model being trained on a plurality of classification features relating to the input DNA mixture.

4. The system according to claim 3, wherein the plurality of classification features comprises at least one of a group comprising:
a plurality of autosomal loci;
estimated concentrations for minor and major contributors;
minor allele counts ratio for each autosomal loci within the input DNA mixture;
number of loci with a minor allele within the input DNA mixture; and
global allele frequencies for each of the plurality of autosomal loci.

5. The system according to claim 3, further comprising applying a threshold responsive to a predicted DNA marker at each genetic location and the estimated concentrations.

6. The system according to claim 3, wherein the supervised learning model includes a random forest model.

7. The system according to claim 6, wherein the random forest model is operated to deconvolve two-person mixtures.

8. The system according to claim 1, wherein the processing component is used within an identification pipeline.

9. The system according to claim 8, wherein the processing component is used to identify and select two-person mixtures for processing through the identification pipeline.

10. The system according to claim 3, wherein the supervised learning model includes at least one output from a group comprising:
a probability for each possible genotype combination contained in the mixture;
a predicted genotype with a highest probability score; and
predicted DNA profiles and corresponding prediction probabilities for each of the at least two DNA contributors.

11. The system according to claim 1, wherein the processing component is configured to deconvolve input DNA mixture comprising at least two DNA contributors into at least two distinct DNA profiles.

12. The system according to claim 11, wherein the processing component is configured to determine the at least two distinct DNA profiles without performing a comparison with one or more DNA reference profiles.

13. The system according to claim 1, wherein the component configured to identify a sex of the at least two DNA contributors further comprises a learning model, the model being trained on a plurality of classification features relating to the input DNA mixture.

14. The system according to claim 13, wherein the plurality of classification features comprises a total number of counts of non-autosomal loci of the input DNA mixture at each sex genetic location.

15. A method comprising:
processing an input DNA mixture,
identifying the number of contributors in the DNA mixture and select mixtures comprising two DNA contributors;
identifying a sex of the two DNA contributors;
identifying a concentration of the two DNA contributors;
determining an individual DNA profile for the two DNA contributors.

16. The method according to claim 15, wherein the one or more forensic genealogy databases comprise DNA markers enabling long-range familial searching of at least three degrees.

17. The method according to claim 15, further comprising training a supervised learning model on a plurality of classification features relating to the input DNA mixture.

18. The method according to claim 17, wherein the plurality of classification features comprises at least one of a group comprising:
a plurality of autosomal loci;
estimated concentrations for minor and major contributors;
minor allele counts ratio for each autosomal loci within the input DNA mixture;
number of loci with a minor allele within the input DNA mixture; and
global allele frequencies for each of the plurality of autosomal loci.

19. The method according to claim 17, further comprising applying a threshold responsive to a predicted DNA marker at each genetic location and the estimated concentrations.

20. The method according to claim 17, wherein the supervised learning model includes a random forest model.

21. The method according to claim 20, wherein the random forest model is operated to deconvolve two-person mixtures.

22. The method according to claim 15, wherein the processing an input DNA mixture is performed within an identification pipeline.

23. The method according to claim 22, wherein the processing an input DNA mixture comprises identifying and selecting two-person mixtures for processing through the identification pipeline.

24. The method according to claim 17, wherein the supervised learning model includes at least one output from a group comprising:
a probability for each possible genotype combination contained in the mixture;
a predicted genotype with a highest probability score; and
predicted DNA profiles and corresponding prediction probabilities for each of the at least two DNA contributors.

25. The method according to claim 15, further comprising: deconvolving input DNA mixture comprising at least two DNA contributors into at least two distinct DNA profiles.

26. The method according to claim 25, further comprising determining the at least two distinct DNA profiles without performing a comparison with one or more DNA reference profiles.

27. The method according to claim 15, wherein the identifying a sex of the two DNA contributors comprises training a learning model on a plurality of classification features relating to the input DNA mixture.

28. The method according to claim 27, wherein the plurality of classification features comprises a total number of counts of non-autosomal loci of the input DNA mixture at each sex genetic location.
